(19) United States
(12) Patent Application Publication
(10) Pub. No.: US 2017/0165089 A1
(43) Pub. Date: Jun. 15, 2017

(54) DEGRADABLE STENT

(71) Applicant: DONGGUAN DIANFU PRODUCT DESIGN CO., LTD., Dongguan (CN)

(72) Inventor: Yangde LI, Dongguan (CN)

(21) Appl. No.: 15/444,223

(22) Filed: Feb. 27, 2017

Related U.S. Application Data
(63) Continuation-in-part of application No. PCT/CN2014/088548, filed on Oct. 14, 2014.

(30) Foreign Application Priority Data
Aug. 29, 2014 (CN) 201410436927.X

(51) Int. Cl. A61F 2/848; A61F 2/04; A61F 2/856

(52) U.S. Cl. CPC A61F 2/848; A61F 2/856; A61F 2/04; A61F 2002/8483; A61F 2210/0004; A61F 2230/0067; A61F 2230/0069; A61F 2230/0052; A61F 2230/006

(57) ABSTRACT

A degradable stent, including: a cylindrical magnesium alloy tube including an outer wall including barbs, and a magnesium alloy wire. In use, the cylindrical magnesium alloy tube is inserted in a broken or damaged lumen of human body, the barbs are inserted in the inner wall of the lumen, the magnesium alloy wire is wound around the magnesium alloy tube and the lumen, and the magnesium alloy tube is fixedly connected to the lumen of the human body.

### CROSS-REFERENCE TO RELATED APPLICATIONS

This application is a continuation-in-part of International Patent Application No. PCT/CN2014/088548 with an international filing date of Oct. 14, 2014, designating the United States, now pending, and further claims foreign priority benefits to Chinese Patent Application No. 201410436927.X filed Aug. 29, 2014. The contents of all of the aforementioned applications, including any intervening amendments thereto, are incorporated herein by reference.

### BACKGROUND OF THE INVENTION

Field of the Invention

The invention relates to the field of medical apparatus and instruments, and more particularly, to a degradable stent.

Description of the Related Art

Conventional magnesium alloy stents are tubular in shape and have smooth surface so that the stents tend to detach from the lumens, or dislocate, or even collapse as the magnesium alloy is absorbed by the human body over time.

### SUMMARY OF THE INVENTION

In view of the above-described problems, it is one objective of the invention to provide a degradable stent which can steadily be implanted in human body.

To achieve the above objective, in accordance with one embodiment of the invention, there is provided a degradable stent, comprising a cylindrical magnesium alloy tube and a magnesium alloy wire. An outer wall of the magnesium alloy tube is provided with a plurality of barbs. The magnesium alloy tube is inserted in a broken or damaged lumen in human body, and the barbs are inserted in an inner wall of the lumen in the human body, thus the stent is connected to the broken and damaged lumen in the human body. The magnesium alloy wire is wound around the magnesium alloy tube and the lumen, thus the magnesium alloy tube is fixedly connected to the lumen in the human body.

In a class of this embodiment, the magnesium alloy tube comprises a main body and at least one branch. The main body communicates with the branch. Sizes and shapes of the main body and the branch are tailored according to actual conditions of the lumen in the human body, for example, the main body and the branch form T-shaped bifurcation, a Y-shaped bifurcation, or an amorphous bifurcation.

In a class of this embodiment, a joint between the main body and the branch is coated with a magnesium alloy protection layer. Bifurcations of the lumens in the human body (including vessel, lymphatic tube, nerve, or biliary tract) are the positions which bear the most frequent blood rush or body fluid rush, thus the magnesium alloy tube placed at the bifurcations is more likely to break or collapse. Therefore, a magnesium alloy protection layer is applied to the magnesium alloy tube which is placed at the bifurcations, ensuring that the magnesium alloy tube always supports the lumens till the lumens are reconstructed, and improving the security of the stent made from magnesium alloy.

In a class of this embodiment, the barbs are disposed on the outer wall of the magnesium alloy tube. The barbs are staggered and opposite-distributed. The magnesium alloy tube is inserted in the lumen of the human body, and the barbs are inserted in a tube wall of the lumen, thus the magnesium alloy tube is connected to the lumen of the human body, and the lumen of the human body is stapled.

In a class of this embodiment, the barbs are conical, bottoms of the conical barbs are integrated with the magnesium alloy tube. The barbs are conical, thus it is convenient for the barbs to be inserted in the tube wall of the lumen of the human body. The barbs are opposite-distributed, thus the tube wall of the broken lumens is tensioned towards a center of the magnesium alloy tube.

In a class of this embodiment, the barbs are not limited to be conical. The barbs can be in other shapes. Optionally, the barbs comprise a cylindrical body and a conical top, and the cylindrical body is integrated with the magnesium alloy tube.

In a class of this embodiment, when the magnesium alloy tube is connected to the lumen of the human body, the magnesium alloy wire operates to fixedly connect the magnesium alloy tube to the lumen, thus preventing the magnesium alloy tube from detaching from the lumen or dislocating.

In a class of this embodiment, the magnesium alloy tube, the magnesium alloy wire, and the barbs are coated with a protection layer. The protection layer is a silver ion layer, or a biocompatible and anti-corrosion coating. The silver ion layer and the biocompatible and anti-corrosion coating feature strong bactericidal ability, prolongs the degradation time of the magnesium alloy, and ensures that the magnesium alloy tube always supports the lumens till the lumens are reconstructed, thus the security of the stent is improved

In a class of this embodiment, a main component of the magnesium alloy tube is magnesium, and the magnesium alloy tube contains iron, zinc, calcium, or a mixture thereof. Magnesium, iron, zinc, and calcium can be absorbed by the human body, thus bring no harm to the human body.

The outer wall of the magnesium alloy tube of the stent is provided with barbs and the magnesium alloy wire. The stent is inserted in the lumen of the human body, and the barbs are inserted in the inner wall of the lumen. The magnesium alloy wire is wound around the magnesium alloy tube and the lumen, thus the magnesium alloy tube is fixedly connected to the lumen of the human body, preventing the magnesium alloy tube from detaching from the lumen of the human body, dislocating, or event collapsing as the magnesium alloy is absorbed by the human body.

Advantages of the degradable stent according to embodiments of the invention are summarized as follows:

The outer wall of the stent is provided with the barbs and the magnesium alloy wire. The magnesium alloy tube is inserted in the lumen of the human body, and the barbs are inserted in the inner wall of the lumen. The magnesium alloy wire is wound around the magnesium alloy tube and the lumen, thus the stent is fixedly connected to the lumen of the human body, preventing the magnesium alloy tube from detaching from the lumen of the human body, dislocating, or event collapsing as the magnesium alloy is absorbed by the human body. A joint between the main body and the branch is coated with a magnesium alloy protection layer, thus ensuring that the stent always supports the lumens till the lumens are reconstructed, and improving the security of the stent made from magnesium alloy.

### BRIEF DESCRIPTION OF THE DRAWINGS

The invention is described hereinbelow with reference to the accompanying drawings, in which:

FIG. 1 is a first schematic diagram of a degradable stent in accordance with one embodiment of the invention; and

FIG. 2 is a second schematic diagram of a degradable stent in accordance with one embodiment of the invention.

### DETAILED DESCRIPTION OF THE EMBODIMENTS

For further illustrating the invention, experiments detailing a degradable stent are described below. It should be noted that the following examples are intended to describe and not to limit the invention.

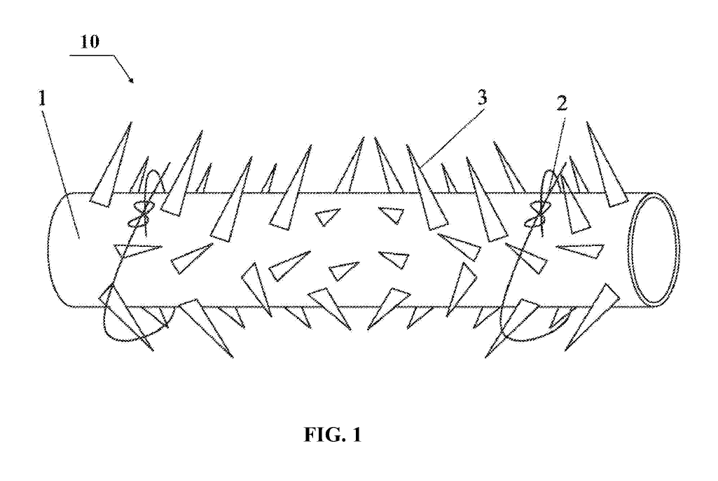

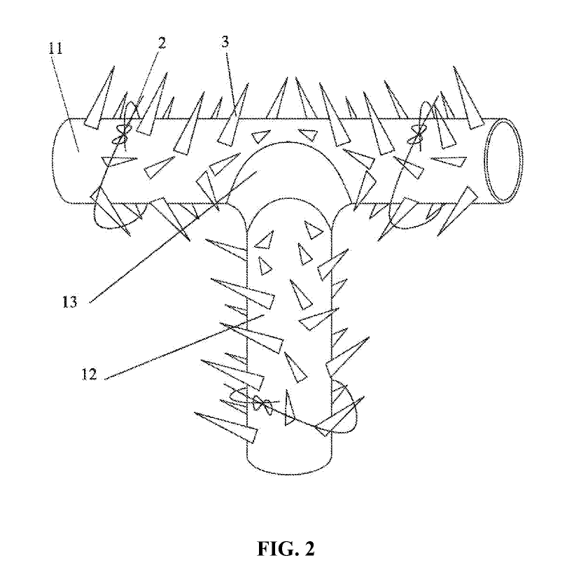

As shown in FIGS. 1-2, a degradable stent 10 comprises a cylindrical magnesium alloy tube 1 and a magnesium alloy wire 2. The outer wall of the magnesium alloy tube 1 is provided with a plurality of barbs 3. The magnesium alloy tube 1 is inserted in a lumen in human body, and the barbs 3 are inserted in an inner wall of the lumen in the human body. The magnesium alloy wire 2 is wound around the magnesium alloy tube 1 and the lumen, thus the stent 10 is fixedly connected to the lumen of the human body, preventing the stent 10 from detaching from the lumen of the human body, dislocating, or event collapsing as the magnesium alloy is absorbed by the human body.

The magnesium alloy tube 1, the magnesium alloy wire 2, and the barbs 3 are coated with a protection layer. The protection layer is a silver ion layer, or a biocompatible and anti-corrosion coating. The magnesium alloy contains iron, zinc, calcium, or a mixture thereof.

As shown in FIG. 2, the magnesium alloy tube comprises a main body 11 and at least one branch 12. The main body 11 communicates with the branch 12. Sizes and shapes of the main body 11 and the branch 12 are tailored according to actual conditions of the lumen in the human body, for example, the main body and the branch form a T-shaped bifurcation, a Y-shaped bifurcation, or an amorphous bifurcation. A joint between the main body 11 and the branch 12 is coated with a magnesium alloy protection layer 13. Bifurcations of the lumens in the human body (including vessel, lymphatic tube, nerve, or biliary tract) are the positions which bear the most frequent blood rush or body fluid rush, thus the magnesium alloy tube placed at the bifurcations is more likely to break or collapse. Therefore, a magnesium alloy protection layer 13 is applied to the magnesium alloy tube which is placed at the bifurcations, ensuring that the magnesium alloy tube always supports the lumens till the lumens are reconstructed, and improving the security of the stent made from magnesium alloy. As shown in FIG. 1-2, the barbs 3 are disposed on the outer wall of the magnesium alloy tube. The barbs are staggered and opposite-distributed. The magnesium alloy tube 1 is inserted in the lumen of the human body, and the barbs 3 are inserted in a tube wall of the lumen, thus the stent 10 is connected to the lumen of the human body, and the lumen of the human body is stapled.

As shown in FIG. 1, the barbs 3 are conical, bottoms of the conical barbs are integrated with the magnesium alloy tube 1. The barbs 3 are conical, thus it is convenient for the barbs to be inserted in the tube wall of the lumen of the human body. The barbs are opposite-distributed, thus the tube wall of the broken lumens is tensioned towards a center of the magnesium alloy tube.

The barbs 3 are not limited to be conical in shape. The barbs can be in other shapes. Optionally, the barbs have a cylindrical body and a conical top, and the cylindrical body is integrated with the magnesium alloy tube.

While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

The invention claimed is:

1. A stent, comprising:
a cylindrical magnesium alloy tube comprising an outer wall comprising barbs; and
a magnesium alloy wire;
wherein
in use, the cylindrical magnesium alloy tube is inserted in a broken or damaged lumen of human body, and the barbs are inserted in an inner wall of the lumen; the magnesium alloy wire is wound around the magnesium alloy tube and the lumen, and the magnesium alloy tube is fixedly connected to the lumen of the human body.

2. The stent of claim 1, wherein the magnesium alloy tube comprises a main body and at least one branch; and the main body communicates with the branch.

3. The stent of claim 2, wherein a joint between the main body and the branch is coated with a magnesium alloy protection layer.

4. The stent of claim 2, wherein the main body and the branch form a T-shaped bifurcation, a Y-shaped bifurcation, or an amorphous bifurcation.

5. The stent of claim 1, wherein the barbs are disposed and staggered on the outer wall of the magnesium alloy tube and heads of the barbs are towards a middle part of the magnesium alloy tube.

6. The stent of claim 2, wherein the barbs are disposed and staggered on the outer wall of the magnesium alloy tube and heads of the barbs are towards a middle part of the magnesium alloy tube.

7. The stent of claim 3, wherein the barbs are disposed and staggered on the outer wall of the magnesium alloy tube and heads of the barbs are towards a middle part of the magnesium alloy tube.

8. The stent of claim 4, wherein the barbs are disposed and staggered on the outer wall of the magnesium alloy tube and heads of the barbs are towards a middle part of the magnesium alloy tube.

9. The stent of claim 5, wherein the barbs are conical, bottoms of the conical barbs are integrated with the magnesium alloy tube.

10. The stent of claim 6, wherein the barbs are conical, bottoms of the conical barbs are integrated with the magnesium alloy tube.

11. The stent of claim 7, wherein the barbs are conical, bottoms of the conical barbs are integrated with the magnesium alloy tube.

12. The stent of claim 8, wherein the barbs are conical, bottoms of the conical barbs are integrated with the magnesium alloy tube.

13. The stent of claim 5, wherein the barbs comprise a cylindrical body and a conical top, and the cylindrical body is integrated with the magnesium alloy tube.

14. The stent of claim 6, wherein the barbs comprise a cylindrical body and a conical top, and the cylindrical body is integrated with the magnesium alloy tube.

15. The stent of claim 7, wherein the barbs comprise a cylindrical body and a conical top, and the cylindrical body is integrated with the magnesium alloy tube.

16. The stent of claim 8, wherein the barbs comprise a cylindrical body and a conical top, and the cylindrical body is integrated with the magnesium alloy tube.

17. The stent of claim 1, wherein the magnesium alloy tube, the magnesium alloy wire, and the barbs are coated with a protection layer; and the protection layer is a silver ion layer, or a biocompatible and anti-corrosion coating.

18. The stent of claim 1, wherein the stent comprises magnesium and at least one of iron, zinc, calcium.
